(19) United States
(12) Patent Application Publication
(10) Pub. No.: US 2024/0024057 A1
(43) Pub. Date: Jan. 25, 2024

(54) SURGICAL INSTRUMENT WITH ELECTROPOLISHED TUNGSTEN CABLE

(71) Applicant: Intuitive Surgical Operations, Inc., Sunnyvale, CA (US)

(72) Inventor: Andrew C. Waterbury, Sunnyvale, CA (US)

(21) Appl. No.: 18/038,414

(22) PCT Filed: Nov. 22, 2021
(86) PCT No.: PCT/US2021/060398

§ 371 Date: May 23, 2023

Related U.S. Application Data
(60) Provisional application No. 63/145,270, filed on Feb. 3, 2021.
(60) Provisional application No. 63/117,397, filed on Nov. 23, 2020.

(51) Int. Cl. A61B 34/00; A61B 34/35

(52) U.S. Cl. CPC A61B 34/71; A61B 34/35; A61B 34/74; A61B 2034/302

(57) ABSTRACT

A surgical instrument includes one or more cables constructed of individual tungsten wires having polished surfaces. As a result, rate of loss of instrument quality of motion over time is significantly reduced, and so instrument usable life is significantly increased.

### CLAIM OF PRIORITY

This application claims the benefit of priority to U.S. Patent Application Ser. No. 63/145,270, filed on Feb. 3, 2021, and to U.S. Patent Application Ser. No. 63/117,397, filed on Nov. 23, 2020, each of which is incorporated by reference herein in its entirety.

### BACKGROUND

Minimally invasive surgical techniques may reduce the amount of damage to tissue during diagnostic or surgical procedures, thereby reducing patient recovery time, discomfort, and unhealthy side effects. A common form of minimally invasive surgery is endoscopy, and a common form of endoscopy is laparoscopy, which is minimally invasive inspection and surgery inside the abdominal cavity. In standard laparoscopic surgery, a patient's abdomen is insufflated with gas, and cannula sleeves are passed through small (approximately one-half inch or less) incisions to provide entry ports for surgical instruments. Other forms of minimally invasive surgery include thoracoscopy, arthroscopy, and similar “keyhole” surgeries that are used to carry out surgical procedures in the abdomen, thorax, throat, rectum, joints, etc.

Teleoperated surgical systems that operate with computer assistance (“telesurgical systems”) are known. These surgical systems are used for both minimally invasive surgeries, and also for “open” surgeries in which an incision is made sufficiently large to allow a surgeon to directly access a surgical site. Examples of minimally invasive and open surgeries include the surgeries listed above, as well as surgeries such as neurosurgery, joint replacement surgery, vascular surgery, and the like, using both rigid- and flexible-shaft teleoperated surgical instruments.

Teleoperated surgical systems often use interchangeable surgical instruments that include end effectors and are controlled by user-commanded robotic manipulator technology. Some instrument types are designed for use in multiple surgical procedures involving different patients, which requires cleaning and sterilizing between procedures. An advantage of multiple-use instruments is that the instrument cost per surgical procedure is reduced. But mechanical and material constraints, such as cable wear and damage that naturally occurs during normal use, limit the number of times these multiple-use instruments can be used. Thus, there is a need to reduce the rate of cable wear and damage during normal use to increase the number of times that a multiple-use instrument can be used.

### SUMMARY

A surgical instrument includes one or more cables constructed of individual tungsten wires having polished surfaces. A surgical instrument with cables made from polished wires unexpectedly and surprisingly sustains quality of motion over multiple use cycles better than an instrument with cable with as-drawn wire (i.e., wire that is not polished). A surgical instrument includes a shaft having a proximal end and a distal end. A movable end effector is coupled to the distal end of the shaft. A drive transmission structure such as a capstan is coupled to the proximal end of the shaft. A drive connector that includes one or more cables is coupled between the drive transmission structure and the end effector. At least one of the one or more cables comprises a plurality of individual tungsten wires. Each wire has a polished outer surface.

Cables within a surgical instrument ordinarily are subjected to tension to achieve high quality instrument motion. Polished wires do not have an oxide layer that is thick enough such that wearing off the oxide layer over time can result in sufficient thinning of the wire diameters and corresponding lengthening of cable, which causes increased slack and loss of tension. Loss of tension over time can result in reduced quality of instrument motion. Thus, polishing of tungsten wire results in reduced rate of loss of tension over time, which results in reduced rate of loss of quality of motion over time.

### BRIEF DESCRIPTION OF DRAWINGS

Aspects of the present disclosure are best understood from the following detailed description when read with the accompanying figures. It is emphasized that, in accordance with the standard practice in the industry, various features are not drawn to scale. In fact, the dimensions of the various features may be arbitrarily increased or reduced for clarity of discussion. In addition, the present disclosure may repeat reference numerals and/or letters in the various examples. This repetition is for simplicity and clarity and does not in itself dictate a relationship between the various embodiments and/or configurations discussed.

FIG. 1 is an illustrative plan view of an example minimally invasive teleoperated surgical system for performing a minimally invasive diagnostic or surgical procedure on a patient who is lying on an operating table.

FIG. 2 is a perspective view of an example user control unit.

FIG. 3 is a perspective view of an example manipulator unit of the minimally invasive teleoperated surgical system of FIG. 1.

FIG. 4A is an illustrative side view of an example surgical instrument.

FIG. 4B is an illustrative functional schematic side view of the example surgical instrument of FIG. 4A.

FIG. 4C is an illustrative drawing showing certain details of the example first drive connector of FIG. 4B.

FIG. 5 is a schematic drawing of an alternative example surgical instrument.

FIG. 6 is an illustrative perspective view of an example known cable.

FIG. 7 is an illustrative cross section view of a first example cable.

FIG. 8 is an illustrative cross section view of a second example cable.

FIG. 9 is an illustrative cross section view of a third example cable.

FIGS. 10A-10B are illustrative perspective, partially cut away, views of a pivotable wrist portion of a surgical instrument that mounts an articulable jaw end effector, shown in two different positions.

FIG. 11 is an illustrative drawing showing curves representing experimental results for a first experiment.

FIG. 12 is an illustrative example mechanical schematic view of an instrument and corresponding axes or rotation of components thereof to show quality of motion in multiple degrees of freedom.

FIG. 13 is an illustrative drawing showing curves representing experimental results for a second experiment.

### DETAILED DESCRIPTION

The inventor unexpectedly and surprisingly found that a surgical instrument that uses polished tungsten wire cable has increased useful surgical instrument life as measured in terms of quality of end effector motion over time. A surgical instrument includes multiple movable components that can degrade from use. For safety, therefore, a telesurgical system typically limits the number of times a surgical instrument may be used. For example, an instrument design typically may be tested to determine expected average maximum life, and then a large safety margin is introduced to define a maximum usable life (e.g., number of times the instrument may be used during normal operation, amount of time the instrument may be used during operation, and the like) that is shorter than the expected average maximum life.

One or more cables containing tungsten wires are typically incorporated within a surgical instrument, which ordinarily is discarded after the instrument has reached its maximum useful life. During production, tungsten wires used to construct surgical instrument cables are drawn at a high temperature such that an oxide layer forms on the wires.

Polished wire refers to tungsten wire in which an oxide layer (e.g., formed during high temperature production) is removed through a post-production processing referred to herein as polishing. Post-production polishing techniques include electropolishing and chemical polishing. Electropolished tungsten wire refers to tungsten wire in which the oxide layer formed during high temperature production of the wire is removed through electropolishing. Electropolishing is an electrochemical technique to remove material from a metallic workpiece. Chemical polishing of tungsten wire removes an oxide layer from the wire by using a chemical process in which one or more chemical baths or chambers (which may be at an elevated temperature for more effective processing) are used to create a chemical reaction that strips the oxide layer off of the outside of the wire (e.g., as part of a reel-to-reel process). Although some oxide can form on tungsten wire during normal use following production, such as during instrument cleaning at elevated temperatures, the amount of oxide formed is far less than oxide formed during production because temperatures during normal use are far lower than the oxide-promoting temperatures during drawing of the tungsten wire.

The inventor observed that cable formed of non-polished tungsten wire changed visual appearance, becoming shinier, as a surgical instrument accumulates use cycles. When manipulating the cable in unconventional ways, such as “plucking” the cable like a guitar string, it was also observed that the natural frequency of the cable decreased as it became shinier. These observations motivated the inventor to explore the effect of the surface oxide layer formed on the tungsten wires upon cable performance and upon performance of a surgical instrument employing the cable.

The inventor performed comparative experiments in which fifteen instruments were tested with as-drawn tungsten wire and ten instruments were tested with polished tungsten wire. The experiments measured quality of instrument motion, which can be thought of as consistent and high correlation of motion of an instrument component for a commanded mechanical input. The experiments demonstrated that, surprisingly, a tool using cables formed of polished tungsten wires exhibited more sustained quality of motion over time than a tool using cables formed of non-polished wires.

The inventor currently believes that surgical instrument use cycles, which involve cleaning and sterilizing the surgical instrument followed by a surgical use, may have an effect like the reduction of oxide present, creating the shinier surface and reduced natural frequency that the inventor observed during the unconventional cable manipulation. That is, the use cycles may result in wearing off or loss of some of the oxide layer on each of the many wires that form the cables. The inventor believes that this loss of oxide layer results in thinning of the diameters of individual wires. Cables within a surgical instrument ordinarily are subjected to continual tension to achieve high quality instrument motion, and so the inventor believes the thinning of the individual wire diameters over time results in relative slippage of the individual wires within the strands of a cable, which in turn results in a lengthening of the cables. The inventor believes that the increased cable length results in reduced cable tension, which over time speeds degradation of quality of instrument motion.

That is, a distal surgical instrument component can be moved with precision by using a cable under relatively high tension rather than by using a cable with reduced tension. Reduced tension can result in slack, which allows cable lengthening and small amounts of cable stretch or displacement along the cable's path. Reduced tension and resulting slack can result in reduced quality of motion. This is especially true for a surgical instrument in which multiple cables are used to simultaneously control multiple end effector mechanical degrees of freedom, such as a first cable used to control a first mechanical DOF (e.g., yaw or grip) and a second cable used to control a second DOF (e.g., pitch). The inventor believes that the cables constructed with polished tungsten wires are less susceptible to tension loss, and therefore such cables contribute to more sustained quality of instrument motion over time. The inventor believes the reason for this surprising and unexpected result is that for instruments having cables with polished tungsten wires, the wires have little or no oxide to wear off is during surgical instrument use cycles, and thus, there is less reduction in wire diameter, less wire slippage within cable strands, and less loss of cable tension within the instrument.

### Teleoperated Surgical System

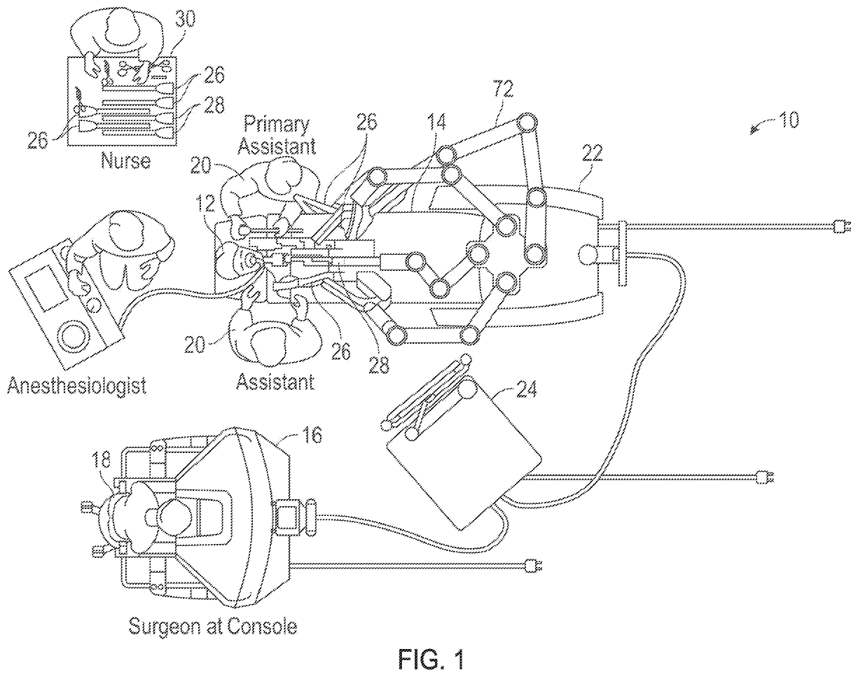

FIG. 1 is an illustrative plan view of an example minimally invasive teleoperated surgical system 10 for performing a minimally invasive diagnostic or therapeutic surgical procedure on a patient 12 who is lying on an operating table 14. The system includes a user control unit 16 for use by a surgeon 18 during the procedure. One or more assistants 20 also may participate in the procedure. The minimally invasive teleoperated surgical system 10 further includes one or more manipulator units 22 and an auxiliary unit computer processing subsystem 24. The manipulator units 22 can manipulate at least one surgical instrument 26 through a minimally invasive incision in the body or a natural body orifice of the patient 12 while the surgeon 18 views the surgical site through the user control unit 16. An image of the surgical site can be obtained by an endoscope 28, such as a stereoscopic endoscope, which may be positioned using a manipulator unit 22. The auxiliary unit 24 includes a computer processing subsystem, which can be centralized or distributed, which can be used to process the images of the surgical site for subsequent display to the surgeon 18 through the user console 16. The auxiliary unit computer processing system 24 includes a logic unit, such as one or more processor circuits, and a memory that stores instructions carried out by the logic unit. In some embodiments, stereoscopic images may be captured, which allow the perception of depth during a surgical procedure. The number of surgical instruments 26 used at one time will generally depend on the diagnostic or therapeutic procedure and the space constraints within the operative site, among other factors. If it is necessary to change one or more of the surgical instruments 26 being used during a procedure, an assistant 20 may remove the surgical instrument 26 from a manipulator unit 22, and replace it with another surgical instrument 26 from a tray 30 in the operating room. An example auxiliary unit computer processing system 24 can be configured to process signals indicative of forces imparted at the surgical instrument. An example auxiliary unit computer processing system 24 can produce signals indicative of haptic feedback corresponding to these imparted forces at the surgeon's console 16. U.S. Pat. No. 6,424,885 B1 (filed Aug. 13, 1999), which is incorporated herein by reference, is an example of a computer control system for a telesurgical system.

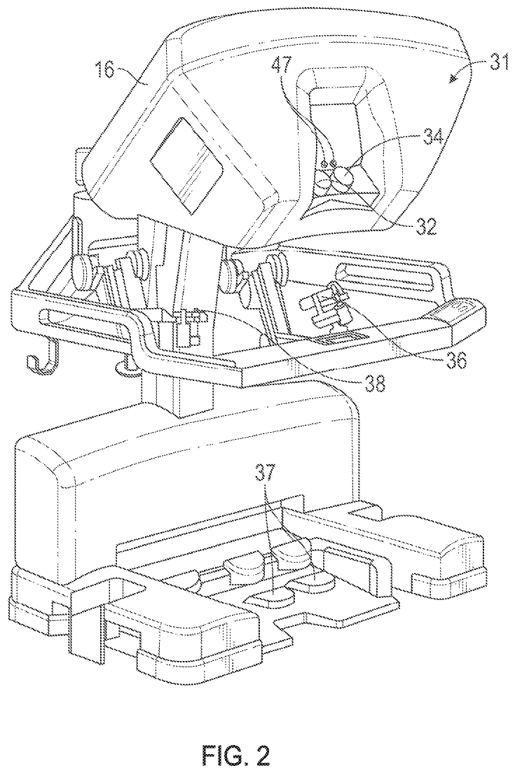

FIG. 2 is a perspective view of an example user control unit 16. The example control unit 16 includes a viewer display 31 that includes a left eye display 32 and a right eye display 34 for presenting the surgeon 18 with a coordinated stereoscopic view of the surgical site that enables depth perception. The control unit 16 further includes one or more hand-operated control input devices 36, 38 to receive larger-scale hand control movements. One or more surgical instruments 26 installed for use at on one or more corresponding manipulator units 22 are operatively coupled to move in relatively smaller-scale distances that match a surgeon 18's larger-scale manipulation of the one or more control inputs 36, 38. In an example system 10, for instance, user (x, y, z) movement is scaled by up to approximately 1:3 to corresponding instrument movement, although the distal DOFs often are not scaled so that the pointing direction of the instrument matches the surgeons hand so that it remains “intuitive”. Thus, in an example system 10 for instance, movement of a control input 36 or 38 by an amount on the order of about a one inch may cause movement of an instrument by an amount on the order of about one third of an inch, for example. In an example system, each control input device 36, 38 is operatively coupled to control a surgical instrument. For example, a first control input device 36 can be operatively coupled to control a first surgical instrument and a second control input device 38 can be operatively coupled to control a second surgical instrument. During a procedure, multiple different surgical instruments can be available at an instrument tray 30, for example, for installation to the manipulator unit 22 for user control via the control unit 16. The control input devices 36, 38 may provide the same mechanical degrees of freedom (DOF) as their associated surgical instruments 26 to provide the surgeon 18 with telepresence, or the perception that respective control input devices 36 are operatively coupled to and integral with the corresponding respective controlled surgical instruments 26 so that the surgeon has a keen sense of directly controlling the instruments 26. To this end, in an example system, position, force, and tactile feedback sensors (not shown) are employed to is transmit position, force, and tactile sensations from the surgical instruments 26 through the control input devices 36, 38 to the surgeon's hands, subject to communication delay constraints. Signals (optionally optical or electronic) modulated based upon forces detected at force sensors (not shown) at the instrument 26 may be processed by the processors at the auxiliary unit cart 24 to produce haptic feedback at the control input devices 36 that is indicative of the detected forces.

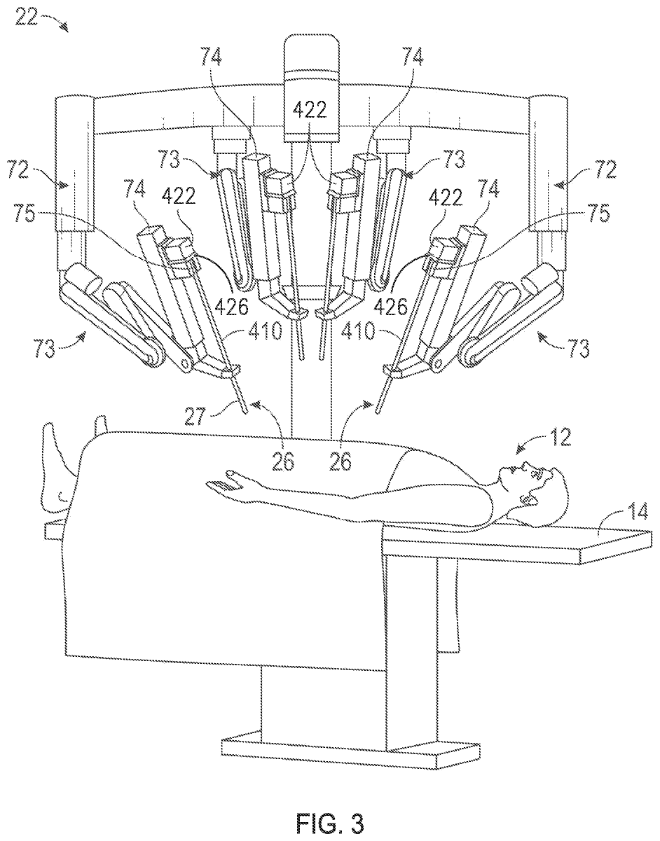

FIG. 3 is a perspective view of an example manipulator unit 22 of the minimally invasive teleoperated surgical system 10. The manipulator unit 22 includes four manipulator support structures 72. Each manipulator support structure 72 includes articulated support structures 73 that are pivotally mounted end-to-end and a pivotally mounted support spar 74. A respective surgical instrument carriage 75, which includes actuators to control instrument motion, is mounted at each support spar 74. Additionally, each manipulator support structure 72 can optionally include one or more setup joints (e.g., unpowered and/or lockable) at the junctions of the articulated support structures 73 and at a junction with a spar 74. A carriage 75 can be moved along a spar 74 to position the carriage 75 at different locations along the spar 74. Thus, the spars 74 can be used to position the attached surgical instrument carriage 75 in relation to a patient 12 for surgery. Each surgical instrument 26 is detachably coupled to a carriage 75. More particularly, a mechanical adapter input interface 426 located between each carriage 75 and each surgical instrument includes drive inputs (not shown) driven by actuators within the carriage 75 that configured to couple rotational torque produced by the actuators to drive elements of the surgical instrument, generally described below. While the manipulator unit 22 is shown as including four manipulator support structures 72, more or fewer manipulator support structures 72 can be used. In general, at least one of the surgical instruments will include a vision system that typically includes an endoscopic camera instrument for capturing video images and one or more video displays for displaying the captured video images that are coupled to one of the carriages 75.

In one aspect, a carriage 75 houses multiple teleoperated actuators (not shown) that impart motion, through the mechanical adapter interface 426, to a tension member, such as a cable drive members, that include drive shafts and capstans (not shown), that in turn drive cable motions that the surgical instrument 26 translates into a variety of movements of an end effector portion of the surgical instrument 26. In some embodiments, the teleoperated actuators in a carriage 75 impart motion to individual components of the surgical instrument 26, such as end effector wrist movement or jaw movement.

A surgeon manipulates the control input devices 36, 38 to control an instrument end effector. An input provided by a surgeon or other medical person to a control input device 36 or 38 (an “input” command) is translated into a corresponding action by the surgical instrument 26 (a corresponding “surgical instrument” response) through actuation of one or more remote actuators. A flexible wire cable-based force transmission mechanism or the like is used to transfer the motions of each of the remotely located teleoperated actuators to a corresponding instrument-interfacing actuator output located at an instrument carriage 75. In some embodiments, a mechanical adapter interface 426 mechanically couples an instrument 26 to drive elements such as drive shafts and capstans (not shown), within an instrument carriage to control motions inside the instrument 26, that in turn, drive cable motions that the surgical instrument 26 translates into a variety of movements of an end effector on the surgical instrument 26.

### Surgical Instrument

The term “surgical instrument” is used herein to describe a medical device for insertion into a patient's body and use in performing a therapeutic or diagnostic procedure. A surgical instrument typically includes moveable component that can include an end effector associated with one or more surgical tasks, such as tissue grasping jaws, a needle driver, shears, a bipolar cauterizer, a tissue stabilizer or retractor, a clip applier, an anastomosis device, an imaging device (e.g., an endoscope or ultrasound probe), and the like. Some surgical instruments used with embodiments further provide an articulated support (sometimes referred to as a “wrist”) for the end effector so that the position and orientation of the end effector can be manipulated with one or more mechanical DOFs in relation to the instrument's shaft 410. Further, many surgical end effectors include a functional mechanical DOF, such as jaws that open or close, or a knife that translates along a path. Surgical instruments appropriate for use in one or more embodiments of the present disclosure may control their end effectors (surgical instruments) with one or more rods and/or flexible cables. In some examples, rods, which may be in the form of tubes, may be combined with cables to provide a pull, push, or combined “push/pull” or “pull/pull” control of the end effector, with the cables providing flexible sections as required. A typical elongated shaft 410 for a surgical instrument is small, for example five to eight millimeters in diameter. The diminutive scale of the mechanisms in the surgical instrument creates unique mechanical conditions and issues with the construction of these mechanisms that are unlike those found in similar mechanisms constructed at a larger scale, because forces and strengths of materials do not scale at the same rate as the size of the mechanisms. The rods and cables must fit within the elongated shaft and be able to control the end effector through the wrist joint. In some example instruments, the cables may be manufactured from a variety of metal (e.g., tungsten or stainless steel) or polymer (e.g., high molecular weight polyethylene) materials.

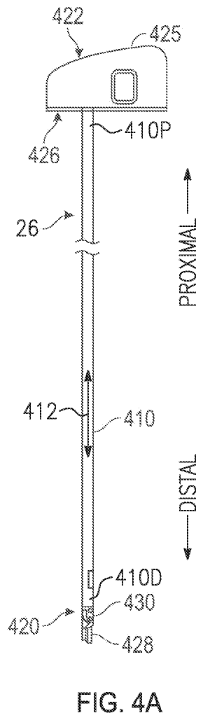

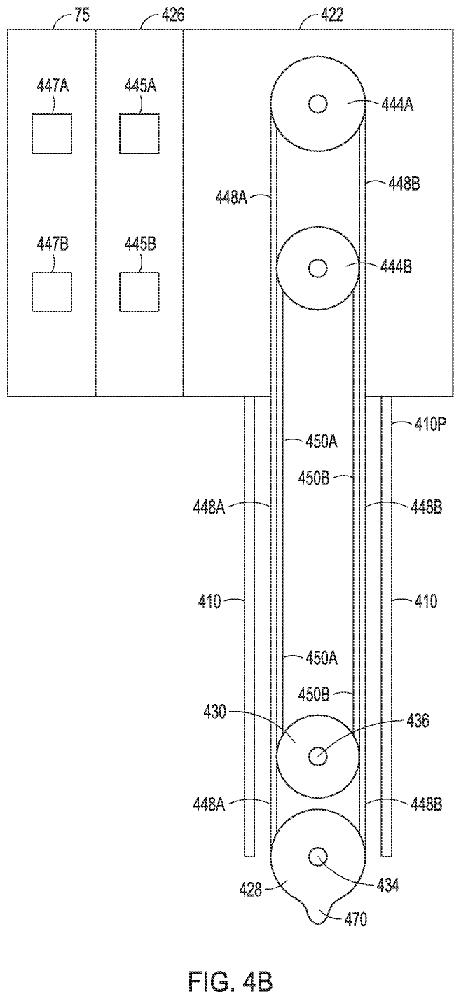

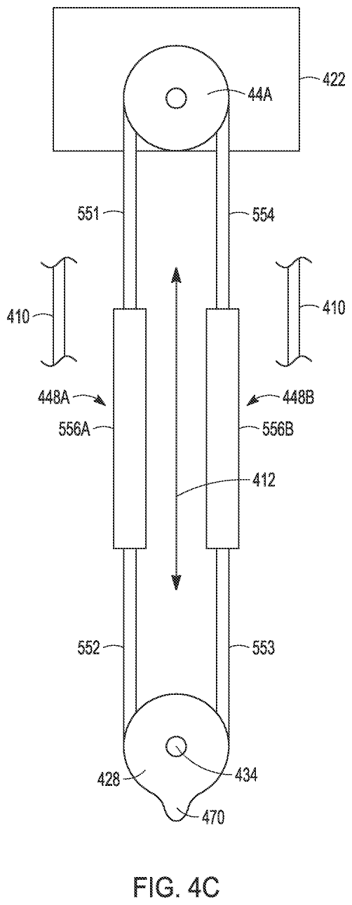

FIG. 4A is an illustrative side elevation view of an example surgical instrument 26 including a shaft defining an internal bore and including a distal portion 410D and a proximal portion 410P. As used herein the term “proximal” indicates a location closer to a manipulator support structure and more distant from a patient anatomy, and the term “distal” indicates a location more distant from the manipulator support structure and closer to the patient. A mechanical structure 422 is coupled to the proximal end portion of the shaft 410. The mechanical structure 422 includes a drive assembly that includes drive elements (not shown) enclosed within a housing 425 used to control movement of a movable component 428 and a wrist 430 located at the distal portion 410D of the shaft 410. The moveable component 428 can include an end effector used to carry out a therapeutic, diagnostic, or an imaging surgical function, or any combination of these functions. For example, the movable component 428 can include any one of a variety of end effectors, such as the jaws, a needle driver, a cautery device, a cutting tool, an imaging device (e.g., an endoscope or ultrasound probe), or a combined device that includes a combination of two or more various instruments and imaging devices. The wrist 430 is coupled at the distal end portion 410D of the shaft, is proximal of the movable component 428, allows the orientation of the moveable component 428 to be manipulated with reference to the elongated shaft 410. Various instrument wrist mechanism configurations are known-see e.g., U.S. Pat. No. 6,394,998 B1 (filed Sep. 17, 1999). U.S. Pat. No. 6,817,974 B2 (filed Jun. 28, 2002), U.S. Pat. No. 9,060,678 B2 (filed Jun. 13, 2007), and U.S. Pat. No. 9,259,275 B2 (filed Nov. 12, 2010), the disclosures of which are incorporated herein by reference. The adapter input interface 426, located between the carriage 75 and the mechanical structure 422 provides a mechanical drive interface between actuators (not shown) within the carriage 75 and the drive elements within the mechanical structure 422 used to drive motion of the. The adapter input interface 426 transmits actuator-driven rotational torques provided by the actuators within the carriage to drive elements within the mechanical structure 422 used to control motion of the movable component 428 and of the wrist joint 430.

FIG. 4B is an illustrative functional schematic side view of the example surgical instrument 26 of FIG. 4A, showing illustrative example functional ranges of motion of the movable component 428 and of the wrist 430. The instrument hollow shaft 410 includes a longitudinal center axis 412 that extends between the proximal end portion 410P and the distal end portion 410D. The movable component 428 is mounted to rotate about a first pin 434 mounted to the distal portion 410D of the shaft and that extends perpendicular to the center axis 412. The wrist joint 430 is mounted to rotate about a second pin 436 mounted to the distal portion 410D of the shaft, proximal to the first pin 434, and that extends perpendicular to the center axis 412. An example shaft 410 is straight. However, alternative example instrument shafts are curved or are jointed.

An example proximal mechanical structure 422 includes one or more drive elements to transmit drive motion to the first and second drive connectors 448, 450, such as rotating disk capstans or various other axially rotating inputs; rotating, rack, or worm gear inputs; lever or gimbal inputs; linear drive elements, such as sliding tab, nut on a lead screw, an element coupled to a fixed position on a cable, and other laterally translating inputs; pin and other axially translating inputs; fluid pressure inputs; and the like. Drive elements of the example surgical instrument 26 of FIG. 4B include first and second rotatable cable drive capstans 444a, 444b located within the housing 425. Drive input discs 445a, 445b, located within the adapter interface 426, transmit torque forces provided by respective actuators 447a, 447b, located within the carriage 75, to drive rotation motion of the respective first and second capstans 444a, 444b. A first drive connector 448 is coupled to impart rotation motion of the movable component 428 about the first pin 434, concurrent with rotation motion of the first drive capstan 444a. A second drive connector 450 is coupled to impart rotation motion of the wrist 430 about the second pin 436, concurrent with rotation motion of the second drive capstan 444b. The first and second drive connectors 448, 450 each includes one or more cables, explained below, that are continually under tension. In an example surgical instrument 26, the first drive capstan 444a is configured before use to continuously impart tension to the one or more cables of the first drive connector 448. Likewise, the second drive capstan 444b is configured before use to continuously impart tension to the one or more cables of the second drive connector 450.

The example first drive connector 448 includes a first drive connector segment 448a and a second drive connector segment 448b. The first and second drive connector segments 448a, 448b each includes a respective proximal cable portion that wraps around the first drive capstan 444a so that a proximal cable portion of the first drive segment 448a pays out and a proximal cable portion of second drive segment 448b pays in as the first capstan 444a rotates in a first direction and so that a proximal cable portion of the first drive segment 448a pays in and a proximal cable portion of second drive segment 448b pays out as the first capstan 444a rotates in a second direction, opposite to the first direction. When a distal end of an instrument is in free space and not in grip, during pay in of the first drive segment 448a and pay out of the second drive segment 448b, the first drive segment 448a imparts a force to move the movable component 428 about the first pin 434, in a direction. Conversely, when a distal end of an instrument is in free space and not in grip, during pay in of the second drive segment 448b and pay out of the first drive segment 448a, the second drive segment 448b imparts a force to move the movable component 428 about the first pin 434, in a direction opposite to the movement direction during pay in of the first drive segment 448a. During pay in and during pay out of the proximal cable portion of the first drive connector segment 448a and during pay in and during pay out of the proximal cable portion of the second drive connector segment 448b, proximal cable portions of both the first and second drive connector segments 448a, 448b are in tension. When, the first and second drive connector segments 448a, 448b also are in tension while at rest, when neither paying in nor paying out. However, it is noted that in some example instruments with jaws, when the instrument jaws are gripping on tissue, for example, the cables driving the jaws together are in tension, but the opposite cables that open the jaws are slack, and do not have tension.

In an alternative example surgical instrument (not shown), a first drive segment is coupled to a first capstan (not shown) and a second drive segment is coupled to second capstan (not shown). In the alternative example surgical instrument, when a distal end of the alternative example instrument is in free space and not in grip, during pay in of a first drive segment about the first capstan and pay out of a second drive segment about the second capstan, the first drive segment imparts a force to move a movable component in a direction. Conversely, when a distal end of an instrument is in free space and not in grip, during pay in of the second drive segment about the second capstan and pay out of the first drive segment about the first capstan, the second drive segment imparts a force to move a movable component about the first pin, in a direction opposite to the movement direction during pay in of the first drive segment.

The example second drive connector 450 includes a third drive connector segment 450a and a fourth drive connector segment 450b. The third and fourth drive connector segments 450a, 450b each includes a respective proximal cable portion that wraps around the second drive capstan 444b so that a proximal cable portion of the third drive segment 450a pays out and a proximal cable portion of the fourth drive segment 450b pays in as the second capstan 444b rotates in a first direction and so that a proximal cable portion of the third drive segment 450a pays in and a proximal cable portion of fourth drive segment 450b pays out as the second capstan 444b rotates in a second direction, opposite to the first direction. When a distal end of an instrument is in free space and not in grip, during pay in of the third drive segment 450a and pay out of the fourth drive segment 450b, the third drive segment 450a imparts a force to move the wrist 430 in a direction. Conversely, when a distal end of an instrument is in free space and not in grip, during pay in of the fourth drive segment 450b and pay out of the third drive segment 450a, the fourth drive segment 450b imparts a force to move the wrist 430 about the second pin 436, in a direction opposite to the movement direction during pay in of the third drive segment 450a. During pay in and during pay out of the proximal cable portion of the third drive connector segment 450a and during pay in and during pay out of the proximal cable portion of the fourth drive connector segment 450b, proximal cable portions of both the third and fourth drive connector segments 450a, 450b are in tension. The third and fourth drive connector segments 450a, 450b also are in tension while at rest, when neither paying in nor paying out. However, as stated above in some example instruments with jaws, when the instrument jaws are gripping on tissue, for example, the cables driving the jaws together are in tension, but the opposite cables that open the jaws are slack, and do not have tension.

In an alternative example surgical instrument (not shown), a third drive segment is coupled to a third capstan (not shown) and a fourth drive segment is coupled to a fourth capstan (not shown). In the alternative example surgical instrument, when a distal end of the alternative example instrument is in free space and not in grip, during pay in of a third drive segment about the third capstan and pay out of a fourth drive segment about the fourth capstan, the third drive segment imparts a force to move a movable component in a direction. Conversely, when a distal end of an instrument is in free space and not in grip, during pay in of the fourth drive segment about the fourth capstan and pay out of the third drive segment about the fourth capstan, the fourth drive segment imparts a force to move a movable component about the first pin, in a direction opposite to the movement direction during pay in of the third drive segment.

FIG. 4C is an illustrative drawing showing certain details of the example first drive connector 448 of FIG. 4B. The first drive connector segment 448a includes a first cable 551, a first rigid element 556a and a second cable 552. In an example first drive connector 448a, the first rigid element 556a includes a first hypotube. The first rigid element 556a is coupled to a distal end portion of the first cable 551 and to a proximal end portion of the second cable 552. A proximal end portion of the first cable 551 is coupled to the first capstan 444a. A distal end portion of the second cable 552 is coupled to the movable component 428, which includes a “tip” portion 470, which is referenced below in the Experiments section. The second drive connector segment 448b includes a third cable 553, a second rigid element 556b and a fourth cable 554. In an example second drive connector 448b, the third rigid element 556b includes a second hypotube. The third rigid element 556b is coupled to a distal end portion of the fourth cable 554 and to a proximal end portion of the third cable 553. A proximal end portion of the fourth cable 554 is coupled to the first capstan 444a. A distal end portion of the third cable 553 is to the movable component 428. Proximal portions of the first cable 551 and the fourth cable 554 wrap around the first drive capstan 444a so that the first cable 551 pays out and the fourth cable 554 pays in as the first capstan 444a rotates in a first direction, and so that the first cable 551 pays in and the fourth cable 554 pays out as the first capstan 444a rotates in a second direction, opposite to the first direction. An example second drive connector 530 has a similar construction that includes four cables and two hypotubes (not shown); for efficiency of description, components of the second drive connector 530 that correspond to components of the first drive connector 528 will not be described again.

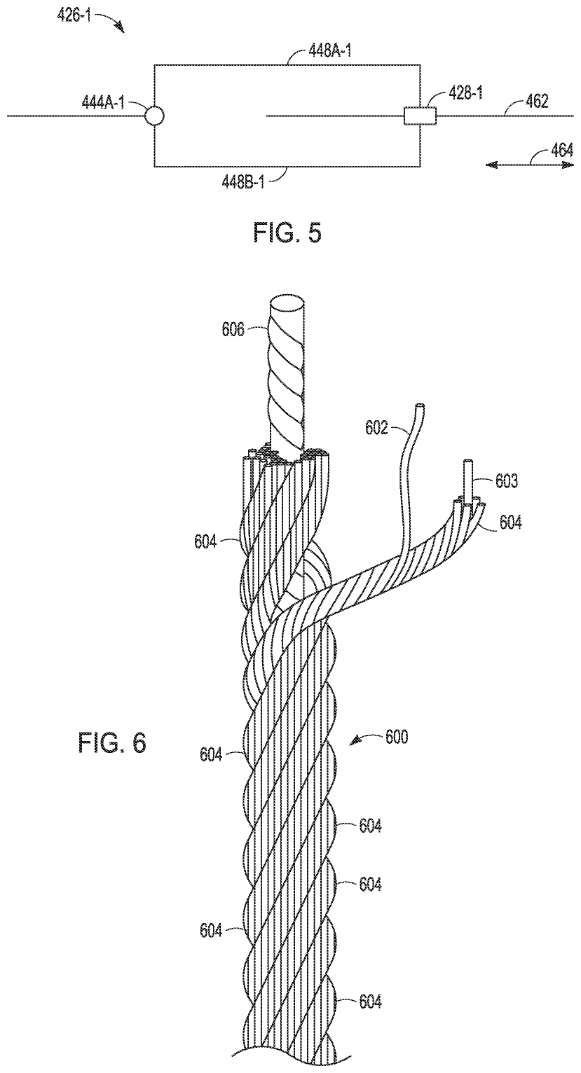

FIG. 5 is an illustrative schematic drawing of an alternative example surgical instrument 426-1 that includes a movable component 428-1 translating at a prismatic joint 462 (e.g., a push rod, a knife blade, a stapler sled, etc.). Double-headed arrow 464 represents a range of motion associated with straight or curvilinear translation constrained by the physical limits of the joint 462. For efficiency of description, example drive connectors 448a-1, 448b-1 and example first capstan drive element 444a-1 of the alternative example surgical instrument 426-1 that correspond to components of the surgical instrument 26 described above with reference to FIGS. 4A-4C will not be described again.

### Cables

A wire cable is a complex intricate machine. Cables generally include three components: a wire, a wire strand, and a core. An example surgical instrument 26 includes cables formed of tungsten. Well-known advantageous properties of tungsten, doped tungsten, and tungsten alloys include strength, high stiffness, high endurance, and resistance to temperature. A wire strand is generally formed by helically winding several wires around a central wire. Several outer strands, in turn, are helically wound about a core to form a complete cable.

FIG. 6 is an illustrative perspective view of an example known cable 600 shown partially unwound that includes multiple stranded wires 602 helically wound about a strand core 603 and that includes multiple strands 604 helically wound about a cable core 606. Wires of an example cable have a diameter in a range 0.025 mm, for example. The cable 600 includes multiple strands. A stranded wire 602 is shown partially unwound from a strand core wire 603, and a strand 604 is shown partially unwound from the cable core 606. The partially unwound strand 604 includes multiple outer wires 602 helically wound about the strand core wire 603. The cable 600 includes multiple strands 604 wound about the core 606. In response to changing stress as the cable 600 is pulled axially and flexed during operation, the helically wound wires 602 within the strands 604 move slightly relative to one another. The strands 604 themselves also slide relative to each other to equalize the more significant stresses within the cable 600. The cable core 606 maintains cable geometry and supports the strands 604 as the wire 602 and strand 604 motions take place, preventing them from collapsing or slipping out of position relative to one another when subjected to radial pressure. As a wire cable 600 is loaded, the helical lay of the strands 604 causes them to press inward toward the cable axis. The core 606 supports this pressure and prevents the strands 604 from rubbing and crushing. The core 606 also maintains the position of the strands 604 during bending.

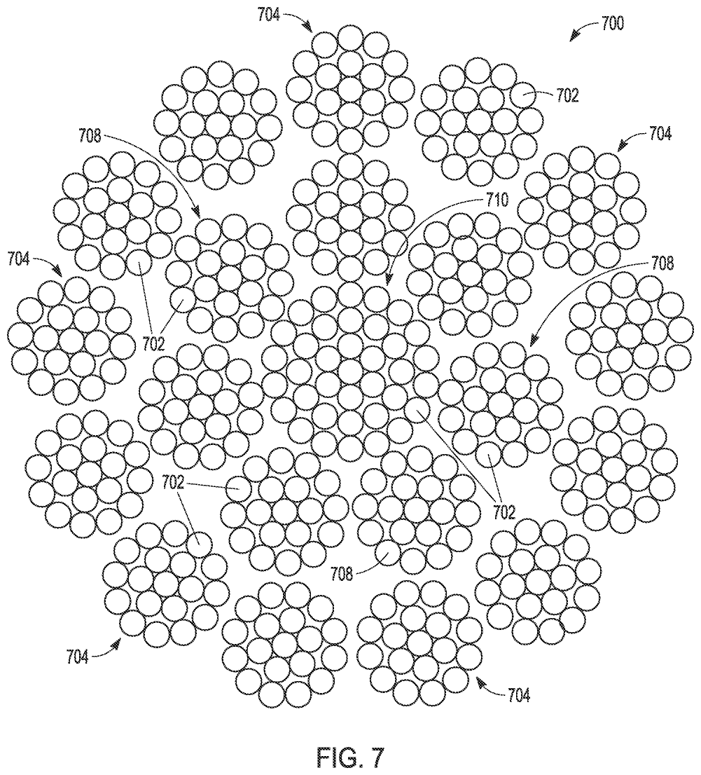

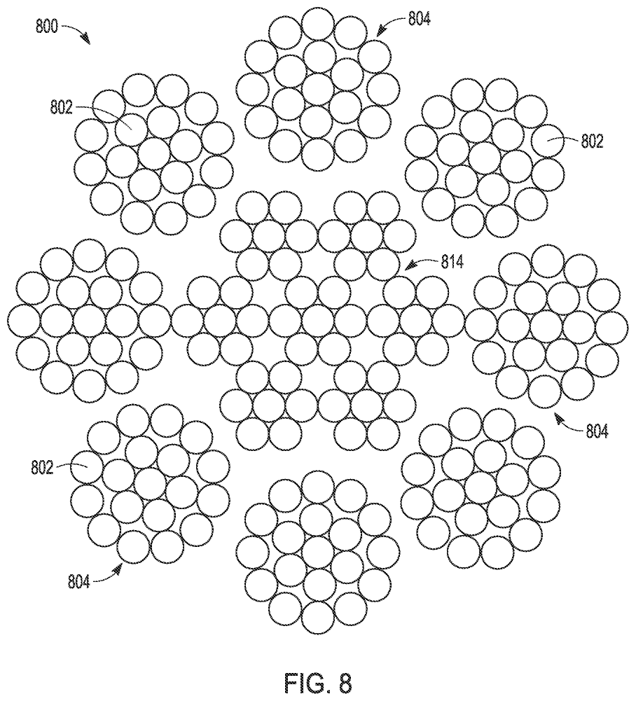

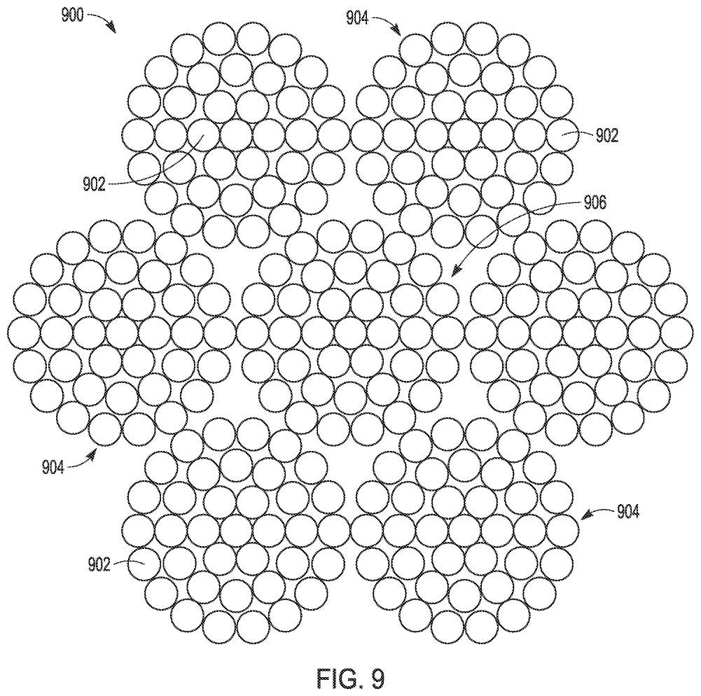

Example cables used within a surgical instrument 26 include a plurality of strands and a multitude of wires arranged in complex configurations. FIG. 7 is an illustrative cross section view of a first cable 700 that has four hundred and seventeen (417) wires 702 arranged in a 13×19-7×19-1×37 construction. The first cable 700 includes thirteen outer strands 704, an inner layer of strands 708 and an inner core 710 The first cable 700 has a wire diameter of approximately 0.015-0.025 mm. FIG. 8 is an illustrative cross section view of a second cable 800 that has two hundred and one (201) wires 802 arranged in an 8×19-7×7 construction. The second cable 800 includes eight outer strands 804 wrapped about a 7×7 core 814. The second cable 800 has a wire diameter of approximately 0.015-0.025 mm. FIG. 9 is an illustrative cross section view of a third cable 900 that has two hundred and fifty-nine (259) wires 902 arranged in a 7×37 construction. The third cable 900 includes six outer strands 904, each having thirty-seven wires. The third cable 900 includes a core region 906 that has a single 1×37 strand. The third cable 900 has a wire diameter of approximately 0.015-0.025 mm.

### Wrist

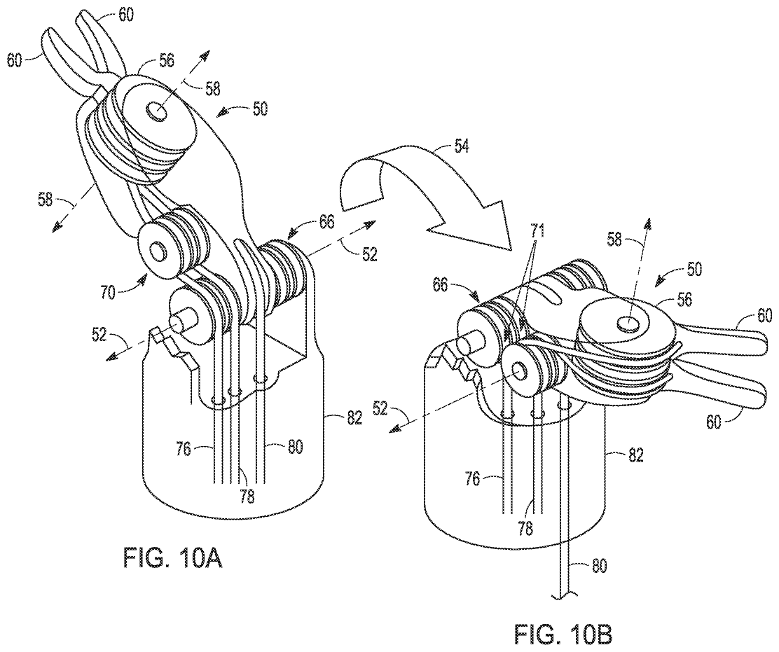

FIGS. 10A-10B are illustrative perspective, partially cut away, views of a pivotable wrist portion 50 of a surgical instrument that mounts an articulable jaw end effector, shown in two different positions. The surgical instrument includes a shaft on which the wrist portion is mounted. The wrist portion includes a first pulley set 70, a second pulley set 66, 71, and a third pulley 74 set to guide first, second and third cable segments 76, 78, 80 that extend from within a shaft 82 and about the pulley sets. The cables 76, 78, 80 are used in combination to cause the wrist portion 50 to pivot about a first axis 52 as indicated by the arrow 54, for example. The cables 76, 78, 80 also are used in combination to cause the end effector portion 56 of the wrist portion 50 to pivot about a second axis 58. The end effector 56 includes jaws 60. It will be appreciated that tensile forces are imparted to the cables 76, 78, 80 as they are used to pull the wrist 50 between pivot positions and as they are used to pivot the end effector 56. Moreover, it will be appreciated that the cables 76, 78, 80 follow a tortuous (i.e. circuitous with sharp curves) paths over several different sets of pulley guide surfaces and that movement of the cables 76, 78, 80 along those paths imparts bending stresses to the cables. It is noted, for example, that cable 76 wraps part way around pulley 66 in a first direction and then wraps part way around pulley 70 in a different direction and then wraps part way around pulley 56 in yet another direction perpendicular to the first direction of motion around pulley 66. Details of an embodiment of the wrist portion 50 of the surgical instrument are provided in U.S. Pat. No. 6,394,998, entitled, “Surgical instruments for Use in Minimally Invasive Telesurgical Applications”.

### Instrument Lifetime

A surgical instrument has a limited useful life. An example surgical instrument has a useful life measured in terms of numbers of cleanings and sterilizations (“CSs”) and number of surgical use (“SUs”). A cleaning and sterilization typically involve hand scrubbing of the distal end of the instrument followed by soaking in an ultrasonic bath of a basic cleaning solution. The ultrasonic bath is followed by an autoclave sterilization that reaches up to 140° C. A surgical use varies depending on the instrument type. For example, a needle driver a surgical use typically involves suturing and knot tying. A typical range of lifetime limit of use of a surgical instrument is ten surgical uses and at least ten cleaning and sterilization cycles.

### Experiment—Lost Motion in Single DOF

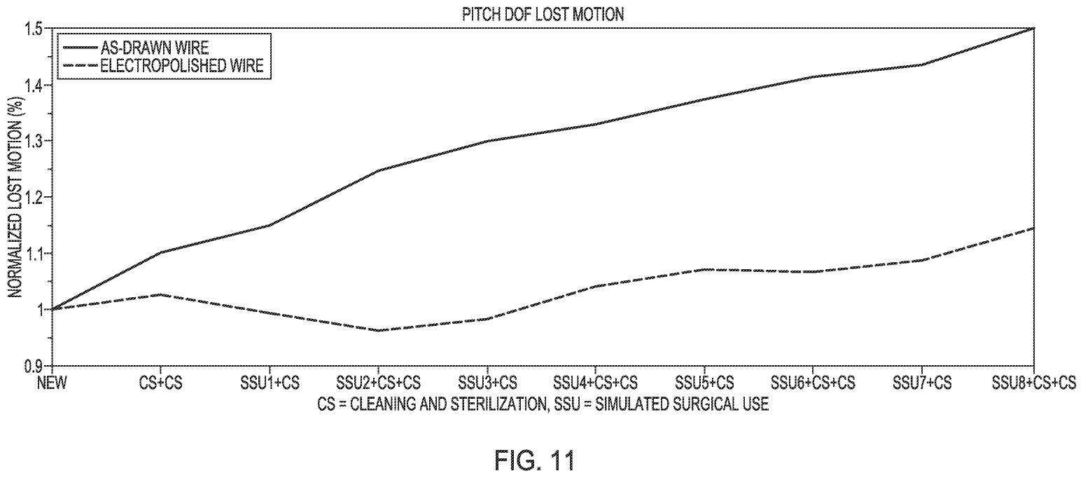

FIG. 11 is an illustrative drawing showing curves representing experimental results for a first experiment involving pitch DOF lost motion versus normalized lost motion for an instrument having cable with as-drawn tungsten wire (“as-drawn wire cable”) and for an instrument having cable with polished tungsten wire (“EP wire cable”). The solid line curve represents the mean experimental results for a population of instruments having as-drawn wire cable. The dashed line curve represents the mean experimental results for a population of tools having EP wire cable. The horizontal axis represents use cycles measured in terms of acts of cleaning and sterilizing (CS) and acts of simulated surgical use (SSU). The vertical axis represents normalized lost motion in terms of degrees per degree. The normalization was achieved by dividing the experiment results by the mean value when the instruments were “new” for each population of instruments. Since the measurement units cancel, the vertical axis provides a dimensionless measure.

The experiment of FIG. 11 involves commanding a instrument to sweep through a pitch motion back and forth along an identical path in forward direction and in a reverse direction, which are opposite one another. In an example instrument, a wrist 430 moves rotationally in a pitch motion about a longitudinal axis of the second pin 436 corresponding to first axis 52 shown in FIGS. 10A-10B. The angular error between the commanded and measured wrist position is recorded in the forward direction and in the reverse direction along the path. The path is rotational, and deviation is measured in terms of degrees of rotation (angles) of the wrist 430 about the second pin 436. Greater angular deviation between the commanded and measured forward and reverse motions along the path signifies increased lost motion. It will be appreciated that lost motion increases with loss of cable tension.

During the experiment, motion of the instrument tip 470 is measured using a two-dimension optical micrometer. A collimated laser beam is shined through a 60 mm round window. Measurements of location of a tip portion 470 of an instrument (e.g., a location of a tip of a movable component) at moments in time are made based upon the shadow cast by the tip. Two-dimensional sampling of tip locations are captured at a fast enough rate to determine deviations from the commanded orientation.

The curves of FIG. 11 show that a surgical instrument that uses EP wire cable sustains quality of instrument motion with increasing use cycles better than an instrument with as-drawn wire cable. For example, referring to use cycle zero (0) on the horizontal axis, curves both the EP wire cable and the as-drawn wire cable are new, the normalized lost motion is one (1), which is the baseline quality of motion for commanded forward and reverse motions for instruments as they come off the manufacturing line. Referring to use cycle SSU1+CS on the horizontal axis, the orientation error deviation for the instrument with the EP wire cable is at about one (1). Whereas, the orientation error deviation for the instrument with the as-drawn wire cable is about 1.25, which signifies approximately twenty-five percent (25%) increase in lost motion. Referring to use cycle SSU4+CS+CS on the horizontal axis, the orientation error deviation for the instrument with the EP wire cable is at about 1.05, which signifies approximately five percent (5%) increase in lost motion. Whereas, the orientation error deviation for the instrument with the as-drawn wire cable is about 1.3, which signifies approximately thirty percent (30%) increase in lost motion. Referring to use cycle SSU8+CS+CS on the horizontal axis, the orientation error deviation for the instrument with the EP wire cable is at about 1.15, which signifies approximately fifteen percent (15%) increase in lost motion. Whereas, the orientation error deviation for the instrument with the as-drawn wire cable is about 1.5, which signifies approximately fifty percent (50%) increase in lost motion. Thus, the experiments of FIG. 11 show that the pitch DOF quality of motion of an instrument with EP wire cable after several use cycles is significantly better than the pitch DOF quality of motion of an instrument with the as-drawn wire cable.

### Example—Multiple DOF Instrument Motion

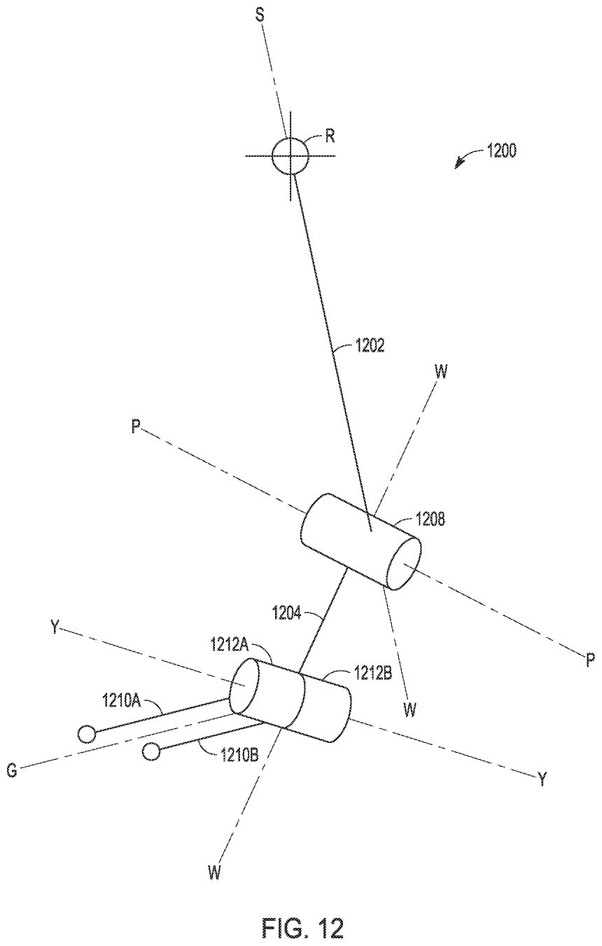

FIG. 12 is an illustrative example mechanical schematic view of an instrument 1200 and corresponding axes or rotation of components thereof to show quality of motion in multiple degrees of freedom. The example instrument 1200 is a representation of a distal portion of a teleoperated ENDOWRIST® surgical instrument commercialized by Intuitive Surgical, Inc., such as a Large Needle Driver instrument. FIG. 12 shows a distal portion of the instrument 1200 that includes an instrument shaft 1202, a wrist link 1204, and first and second grasping jaws 1206a and 1206b. A shaft axis S-S is defined along a length of the instrument shaft 1202. The wrist link 1204 is coupled to instrument shaft 1202 at a revolute wrist joint 1208, which rotates about a pitch axis P-P. A wrist axis W-W is defined along a length of the wrist link 1204. Grasping jaws 1210a, 1210b are coaxially coupled to the wrist link 1204 at corresponding revolute jaw joints 1212a and 1212b, and each rotates about a yaw axis Y-Y. Grasping jaws 1210a, 1210b close at grip axis G. A center of motion R of the instrument 1200 is defined on instrument shaft 1202 and represents a rotational position in space that is to be maintained fixed in space throughout a medical procedure, such as the position at which instrument shaft 1202 enters a patient's body wall.

Quality of motion of the instrument 1200 can be considered as being greater when the instrument can be controlled to maintain a substantially fixed location in space of the center of motion R during complex instrument motion e.g., in multiple degrees of freedom. As the components of instrument 1200, such as cables for example, deteriorate due to use, there is reduced ability to control the instrument to maintain a substantially fixed location in space of the center of motion R during complex instrument motion in multiple degrees of freedom. In other words, as the instrument deteriorates, quality of instrument motion decreases due to a loss of movement precision of instrument components. One cause of loss of movement precision is loss of cable tension. The inventors discovered that instruments with cables having polished wires experience a slower rate of decay of quality of motion than do instruments with cables with as drawn wires. The inventors believe that the reason for the longer lasting quality of motion of instruments with cables with polished wires is that polished wires stretch at a is slower rate and therefore lose tension at a slower rate than do cables with as drawn wires.

During a medical procedure, the clinical user operates computer-assisted teleoperation control inputs 36, 38 to command motions of the instrument 1200 and the instrument's various distal components. One such motion is to roll the grasping jaws 1210a, 1210b about grip axis G, and it can be appreciated that maintaining grip axis G's spatial orientation and position during roll is important for effective instrument control and good clinical performance. Ideally, shaft 1202 rotates about axis S-S, wrist link 1204 rotates about axis W-W, and grasping jaws 1210a, 1210b rotate together about grip axis G, all without any change in orientation or position of grip axis G or center of motion R.

Since cables control simultaneous motions of the wrist link 1204 about pitch axis P-P and the grasping jaws 1210a, 1210b about yaw axis Y-Y as instrument shaft 1202 rolls about axis S-S, effective cable control of each rotational degree of freedom is important to maintain grip axis G's spatial orientation and position. It can be seen that as joints 1208, 1212a, and 1212b are rotated farther from a neutral position (e.g., straight and aligned with shaft axis S-S), such as to grasp and move a suture needle, mechanical tolerances make maintaining grip axis G's spatial position and orientation during instrument roll of the shaft 1202 increasingly challenging. The ability of an instrument to come as close as possible to maintaining ideal roll motion with reference to grip axis G can be thought of as an example of quality of motion of these instrument components.

FIG. 12 shows the distal components of the instrument 1200 with grip axis G displaced in orientation and position with reference to instrument shaft axis S-S by wrist joint 1208 rotation about pitch axis P-P and by the jaw joints' 1212a, 1212b rotations about yaw axis Y-Y. To rotate grasping jaws 1210a, 1210b about grip axis G while keeping instrument shaft 1202 passing through center of motion R, wrist link 1204 must rotate about its longitudinal axis W-W, and at the same time wrist axis W-W will sweep along the surface of an imaginary cone (not shown) with an apex at yaw axis Y-Y between jaw joints 1212a, 1212b. Likewise, instrument shaft 1202 must rotate about its longitudinal axis S-S, and at the same time axis S-S will sweep along the surface of the imaginary cone (not shown) with its apex at center of motion R. (Instrument shaft 1202 may translate through center of motion R as necessary.) As instrument components such as cables, for example, mechanically deteriorate over time, however, the complex interactions among the various cable-controlled mechanical DOFs will cause deviations in the motions of wrist link 1204 and grasping jaws 1210a, 1210b about their associated axes such that grip axis G will no longer stay stationary in space, either in orientation or position. The amount of deviation over time of grip axis G from its ideal, stationary location during a commanded roll motion of grasping jaws 1210a, 1210b about grip axis G may be thought of as an indication of an increasing degradation in motion quality over time. For example, grip axis G may begin to sweep along an irregular conic surface (not shown) that translates in space, and such a sweep motion may become irregular and jerky. Ultimately motion quality will degrade to the point that the instrument becomes unsatisfactory for clinical use and must be replaced. Motion quality can be measured by observing the paths followed by one or more instrument components. In an example instrument 1200, smooth conical paths are indicative of high-quality motion, and irregular or jerky conical paths are indicative of lesser quality motion.

### Experiment—Lost Motion in Multiple DOFs

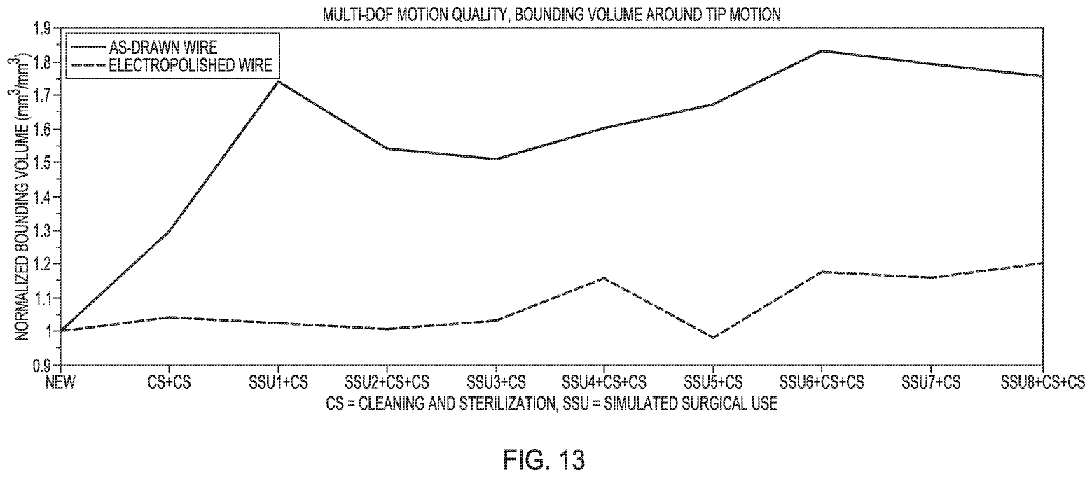

FIG. 13 is an illustrative drawing showing curves representing experimental results for a second experiment involving multi-DOF motion quality bounding volume around instrument tip versus normalized lost motion for an instrument with as-drawn wire cable and for the tool with the EP wire cable. The solid line curve represents experimental results for an instrument having as-drawn wire cable. The dashed line curve represents experimental results for an instrument having EP wire cable.

The experiment of FIG. 13 involves commanding an instrument such as the example instrument 1200 of FIG. 12 to move through a complex six-DOF motion throughout which the instrument tip portion is to be maintained in a fixed position. An example six-DOF motion is to “throw-a-needle”, which is a motion executed by some instruments during a suturing task during a surgical procedure. An objective during a suturing task is to guide a curved needle on a path similar to the curve of the needle between target entry and exit points in the tissue. This motion is analogous to maintaining the instrument tip 470 in a substantially fixed location in space as it rotates about axis G while other components of the instrument move about in three-dimensional space.

The vertical axis in FIG. 13 indicates a normalized bounding volume traversed by the tip during the six-DOF motion. During the six-DOF motion, motion of the tip 470 is measured using two orthogonal two-dimensional vision systems. Based upon the measurements, a bounding volume that encompasses motion of the tip during the six-DOF motion. The larger the bounding volume, the more the tip 470 changed location during the six-DOF motion. A larger bounding volume signifies more lost motion. It will be appreciated that lost motion increases with the loss of cable tension.

The curves of FIG. 13 show that a surgical instrument that uses EP wire cable sustains quality of instrument motion for increasing use cycles better than an instrument with as-drawn wire cable. For example, referring to use cycle zero (0) on the horizontal axis, both the EP wire cable and the as-drawn wire cable are new, the normalized lost motion is one (1), which is the baseline for the motion of the tip portion 470. Referring to use cycle SSU1+CS on the horizontal axis, the normalized bounding volume for an instrument with the EP wire cable is at about one (1). Whereas, the normalized bounding volume for a tool with the as-drawn wire cable is about 1.75, which signifies that the mean bounding volume increased by about 75% from the mean volume of a “new” instrument. Referring to use cycle SSU4+CS+CS on the horizontal axis, the normalized bounding box volume for an instrument with the EP wire cable is at about 1.1, which signifies that the mean bounding volume increased by about 10% from the mean volume of a “new” instrument. Whereas, the normalized bounding volume for an instrument with the as-drawn wire cable is about 1.5, which signifies that the mean bounding volume increased by about 50% from the mean volume of a “new” instrument. Referring to use cycle SSU8+CS+CS on the horizontal axis, the normalized bounding volume for an instrument with the EP wire cable is at about 1.2, which signifies that the mean bounding volume increased by about 20% from the mean volume of a “new” instrument. Whereas, the normalized bounding volume for a tool with the as-drawn wire cable is about 1.75, which signifies that the mean bounding volume increased by about 75% from the mean volume of a “new” instrument. Thus, the experiments of FIG. 13 also show that quality of motion of an instrument with EP wire cable after several use cycles is significantly better than quality of motion of an instrument with the as-drawn wire cable.

The above description is presented to enable any person skilled in the art to create and use a surgical instrument having cables containing polished tungsten wires and corresponding cables containing polished tungsten wires. Various modifications to the embodiments will be readily apparent to those skilled in the art, and the generic principles defined herein may be applied to other embodiments and applications without departing from the scope of the invention. In the preceding description, numerous details are set forth for the purpose of explanation. However, one of ordinary skill in the art will realize that the embodiments in the disclosure might be practiced without the use of these specific details. In other instances, well-known processes are shown in block diagram form in order not to obscure the description of the invention with unnecessary detail. Identical reference numerals may be used to represent different views of the same or similar item in different drawings. Thus, the foregoing description and drawings of examples in accordance with the present invention are merely illustrative of the principles of the invention. Therefore, it will be understood that various modifications can be made to the embodiments by those skilled in the art without departing from the scope of the invention, which is defined in the appended claims.

1. A surgical instrument comprising:
a shaft comprising a proximal end and a distal end;
a moveable component coupled to the distal end of the shaft;
a mechanical structure coupled to the proximal end of the shaft,
the mechanical structure comprising a drive element; and
a drive connector coupled between the drive element and the moveable component, the drive connector comprising one or more cables,
at least one of the one or more cables comprising a plurality of individual wires,
each wire of the plurality of individual wires comprising tungsten, and
each wire of the plurality of individual wires having a polished outer surface.

2. The surgical instrument of claim 1, wherein:
the cable comprises a plurality of strands; and
one or more of the plurality of strands comprises the plurality of individual wires.

3. The surgical instrument of claim 1, wherein:
the cable comprises a core strand and a plurality of outer strands surrounding the core strand; and
the core strand comprises the plurality of individual wires.

4. The surgical instrument of claim 1, wherein:
the cable comprises a core strand and a plurality of outer strands surrounding the core strand; and
one or more strands of the plurality of strands comprises the plurality of individual wires.

5. The surgical instrument of claim 1, wherein:
the cable comprises a core strand and a plurality of outer strands surrounding the core strand; and
the core strand and the plurality of outer strands comprise the plurality of individual wires.

6. The surgical instrument of any one of claim 1, wherein:
each wire of the plurality of individual wires consists essentially of tungsten, doped tungsten, or a tungsten alloy.

7. (canceled)

8. The surgical instrument of claim 1, wherein:
each wire of the plurality of individual wires has a diameter smaller than 0.175 mm.

9. (canceled)

10. The surgical instrument of claim 1, wherein:
the cable has a diameter smaller than 2.0 mm.

11. The surgical instrument of claim 1, wherein:
the drive connector comprises a hypotube;
the hypotube comprises a proximal end; and
the cable is coupled between the proximal end of the hypotube and the drive element.

12. The surgical instrument of claim 1, wherein:
the drive connector comprises a hypotube;
the hypotube comprises a distal end; and
the cable is coupled between the distal end of the hypotube and the moveable component.

13. The surgical instrument of claim 1, wherein:
the drive connector comprises a first hypotube and a second hypotube, the first hypotube comprising a proximal end and a distal end, and the second hypotube comprising a proximal end and a distal end;
the one or more cables comprise a first cable, a second cable, a third cable, and a fourth cable;
the first cable is coupled between the drive element and the proximal end of the first hypotube;
the second cable is coupled between the distal end of the first hypotube and the movable component;
the third cable is coupled between the drive element and the proximal end of the second hypotube; and
the fourth cable is coupled between the distal end of the second hypotube and the movable component.

14. The surgical instrument of claim 1, wherein:
the drive connector comprises a first hypotube and a second hypotube, the first hypotube comprising a proximal end and a distal end, and the second hypotube comprising a proximal end and a distal end;
the one or more cables comprise a first cable, a second cable, and a third cable;
the first cable is coupled between the drive element and the proximal end of the first hypotube;
the second cable is coupled between the distal end of the first hypotube and the distal end of the second hypotube;
the movable component is coupled to the second cable between the distal end of the first hypotube and the distal end of the second hypotube; and
the third cable is coupled between the drive element and the proximal end of the second hypotube.

15. The surgical instrument of claim 1, wherein:
the drive connector comprises a first hypotube and a second hypotube, the first hypotube comprising a proximal end and a distal end, and the second hypotube comprising a proximal end and a distal end;
the one or more cables comprise a first cable, a second cable, a third cable, and a fourth cable;
the mechanical structure comprises a second drive element;
the first cable is coupled between the drive element and the proximal end of the first hypotube;
the second cable is coupled between the distal end of the first hypotube and the movable component;
the third cable is coupled between the second drive element and the proximal end of the second hypotube; and
the fourth cable is coupled between the distal end of the second hypotube and the movable component.

16. The surgical instrument of claim 1, wherein:
the drive connector comprises a first hypotube and a second hypotube, the first hypotube comprising a proximal end and a distal end, and the second hypotube comprising a proximal end and a distal end;
the one or more cables comprise a first cable, a second cable, and a third cable;
the mechanical structure comprises a second drive element;
the first cable is coupled between the drive element and the proximal end of the first hypotube;
the second cable is coupled between the distal end of the first hypotube and the distal end of the second hypotube;
the movable component is coupled to the second cable between the distal end of the first hypotube and the distal end of the second hypotube; and
the third cable is coupled between the second drive element and the proximal end of the second hypotube.

17-21. (canceled)

22. The surgical instrument of claim 1, wherein:
during a first state of the drive connector, the drive connector is stationary;
during a second state of the drive connector, the drive connector is urged by the drive element to translate in a first direction; and
the one or more cables are in tension during the first and second states of the drive connector.

23. The surgical instrument of claim 22, wherein:
the first and second states of the drive connector exist on the condition that the cable has been subjected to ten or more surgical, cleaning, and autoclave sterilization cycles.

24. The surgical instrument of claim 22, wherein:
the first and second states of the drive connector exist on the condition that the cable has been subjected to twenty or more surgical, cleaning, and autoclave sterilization cycles.

25. The surgical instrument of claim 1, wherein:
during a first state of the drive connector, the drive connector is stationary;
during a second state of the drive connector, the drive connector is urged by the drive element to translate in a first direction;
during a third state of the drive connector, the drive connector is urged by the drive element to translate in a second direction opposite the first direction; and
the one or more cables are in tension during the first, second, and third states of the drive connector.

26. The surgical instrument of claim 25, wherein:
the first, second, and third states of the drive connector exist after the cable has been subjected to ten or more surgical, cleaning, and autoclave sterilization cycles.

27. The surgical instrument of claim 25, wherein:
the first, second, and third states of the drive connector exist after the cable has been subjected to twenty or more surgical, cleaning, and autoclave sterilization cycles.

28-30. (canceled)
